(19) United States
(12) Patent Application Publication
(10) Pub. No.: US 2025/0332382 A1
(43) Pub. Date: Oct. 30, 2025

(54) DEVICES TO AID TARGETED DELIVERY OF INJECTATES

(71) Applicants: Boston Scientific Scimed, Inc., Maple Grove, MN (US); MAYO FOUNDATION FOR MEDICAL EDUCATION AND RESEARCH, Rochester, MN (US)

(72) Inventors: Ajay Gupta, Minneapolis, MN (US); Mark Steven Smith, Coon Rapids, MN (US); Beau Bosko Toskich, Ponte Vedra Beach, FL (US)

(21) Appl. No.: 19/186,015

(22) Filed: Apr. 22, 2025

Related U.S. Application Data
(60) Provisional application No. 63/639,247, filed on Apr. 26, 2024.

(51) Int. Cl. A61M 25/00; A61M 25/09

(52) U.S. Cl. CPC A61M 25/007; A61M 25/09; A61M 2025/0042; A61M 2025/0073; A61M 2206/20; A61M 2210/12

(57) ABSTRACT

Embodiments herein relate to dispersion catheter systems and related methods. In an embodiment, a dispersion catheter system for delivering injectates to small diameter vessels of a patient is included having a dispersion catheter shaft defining a lumen and a dispersion catheter tip in fluid communication with the lumen of the dispersion catheter shaft. The dispersion catheter system can also include a fluid dispersal cap, wherein the fluid dispersal cap is configured to be disposed within a path of fluid flow coming from the dispersion catheter tip. The fluid dispersal cap can have one or more internal flow conduits, side flow ports, side flow channels, and/or outflow ports. Other embodiments are also included herein.

This application claims the benefit of U.S. Provisional Application No. 63/639,247, filed Apr. 26, 2024, the content of which is herein incorporated by reference in its entirety.

### FIELD

Embodiments herein relate to dispersion catheter systems and related methods.

### BACKGROUND

In the course of diagnosis and/or treatment of patients, various compositions (injectates) can be administered to the patient by injecting the same into a vessel of the body. The injectate may be a diagnostic or therapeutic composition and carry dissolved or suspended substances including, but not limited to, dyes, contrast agents, radioactive beads, therapeutic particulates, embolic agents, stem cells, oncolytic viruses, immune-active substances, macrophages, chemotherapeutic agents, other active agents, and the like.

However, there are many potential challenges associated with delivering such compositions including limited vessel sizes and runway (depending on the target), downstream branching (which could result in delivery to non-target areas), vessel branches in sequence (series), differing flow resistance in some areas, irregular and/or undesirable deposition patterns of composition components, sedation requirements, vasospasm, and catheter positioning inconsistency.

### SUMMARY

Embodiments herein include devices that can enhance dispersion of an injectate with the blood stream. Embodiments of devices herein can also reduce reflux flow. For these and other reasons, devices herein can make interventional treatments safer and more effective. They can also enable new interventional treatments that are currently difficult and/or contraindicated due to an inability to precisely deliver therapeutics to target anatomies.

In a first aspect, a dispersion catheter system for delivering injectates to small diameter vessels of a patient can be included having a dispersion catheter shaft defining a lumen, a dispersion catheter tip in fluid communication with the lumen of the dispersion catheter shaft, and a fluid dispersal cap, wherein the fluid dispersal cap can be configured to be disposed within a path of fluid flow coming from the dispersion catheter tip.

In a second aspect, in addition to one or more of the preceding or following aspects, or in the alternative to some aspects, the dispersion catheter shaft can have an outside diameter of less than 0.039 in (1 mm).

In a third aspect, in addition to one or more of the preceding or following aspects, or in the alternative to some aspects, the fluid dispersal cap can be positioned at least partially inside of an outflow orifice.

In a fourth aspect, in addition to one or more of the preceding or following aspects, or in the alternative to some aspects, the fluid dispersal cap can be positioned fully outside of the dispersion catheter tip.

In a fifth aspect, in addition to one or more of the preceding or following aspects, or in the alternative to some aspects, the fluid dispersal cap can be circular or semicircular in cross-section.

In a sixth aspect, in addition to one or more of the preceding or following aspects, or in the alternative to some aspects, the fluid dispersal cap can include a proximal end wall, a distal end wall, and a side wall, wherein the side wall interconnects the proximal end wall and the distal end wall.

In a seventh aspect, in addition to one or more of the preceding or following aspects, or in the alternative to some aspects, the side wall can define one or more side flow ports.

In an eighth aspect, in addition to one or more of the preceding or following aspects, or in the alternative to some aspects, the one or more side flow ports can be configured to direct a flow of fluid at least partly in a radial direction of the fluid dispersal cap.

In a ninth aspect, in addition to one or more of the preceding or following aspects, or in the alternative to some aspects, the side wall can define one or more side flow channels.

In a tenth aspect, in addition to one or more of the preceding or following aspects, or in the alternative to some aspects, the proximal end wall can include one or more inflow ports.

In an eleventh aspect, in addition to one or more of the preceding or following aspects, or in the alternative to some aspects, the one or more inflow ports can be centrally positioned on the proximal end wall.

In a twelfth aspect, in addition to one or more of the preceding or following aspects, or in the alternative to some aspects, the one or more inflow ports can be positioned around the periphery of the proximal end wall.

In a thirteenth aspect, in addition to one or more of the preceding or following aspects, or in the alternative to some aspects, the distal end wall can define one or more outflow ports.

In a fourteenth aspect, in addition to one or more of the preceding or following aspects, or in the alternative to some aspects, the one or more outflow ports can be distributed around the periphery of the distal end wall.

In a fifteenth aspect, in addition to one or more of the preceding or following aspects, or in the alternative to some aspects, the one or more outflow ports contact a peripheral edge of the distal end wall.

In a sixteenth aspect, in addition to one or more of the preceding or following aspects, or in the alternative to some aspects, the distal end wall does not include an outflow port therein.

In a seventeenth aspect, in addition to one or more of the preceding or following aspects, or in the alternative to some aspects, the fluid dispersal cap can define one or more internal flow conduits.

In an eighteenth aspect, in addition to one or more of the preceding or following aspects, or in the alternative to some aspects, the one or more internal flow conduits can be curved.

In a nineteenth aspect, in addition to one or more of the preceding or following aspects, or in the alternative to some aspects, the one or more internal flow conduits can be at least partly helical.

In a twentieth aspect, in addition to one or more of the preceding or following aspects, or in the alternative to some aspects, the fluid dispersal cap can be configured to be attached to the dispersion catheter shaft.

In a twenty-first aspect, in addition to one or more of the preceding or following aspects, or in the alternative to some aspects, can further include a delivery wire, wherein the fluid dispersal cap can be configured to be attached to a distal end of the delivery wire and the delivery wire can be configured to pass through the lumen of the dispersion catheter shaft.

In a twenty-second aspect, in addition to one or more of the preceding or following aspects, or in the alternative to some aspects, the dispersion catheter tip can define an outflow orifice and the fluid dispersal cap can be positioned at least partially outside of the outflow orifice.

In a twenty-third aspect, in addition to one or more of the preceding or following aspects, or in the alternative to some aspects, the fluid dispersal cap can be conical in shape.

In a twenty-fourth aspect, a method of delivering an injectate to a small diameter vessel of a patient can be included. The method can include inserting a microcatheter into a vessel of the patient and advancing a dispersion catheter to an end of the microcatheter using a pusher wire. The dispersion catheter can include a fluid dispersal cap. The method can further include removing the pusher wire from the dispersion catheter and delivering a therapeutic composition to the patient through the dispersion catheter.

In a twenty-fifth aspect, in addition to one or more of the preceding or following aspects, or in the alternative to some aspects, the microcatheter can have an inner diameter from 0.021 to 0.027 inches (0.53 to 0.68 mm).

In a twenty-sixth aspect, in addition to one or more of the preceding or following aspects, or in the alternative to some aspects, the dispersion catheter can have an outside diameter of less than 0.039 in (1 mm).

In a twenty-seventh aspect, in addition to one or more of the preceding or following aspects, or in the alternative to some aspects, the fluid dispersal cap can include a proximal end wall, a distal end wall, and a side wall, wherein the side wall interconnects the proximal end wall and the distal end wall.

In a twenty-eighth aspect, in addition to one or more of the preceding or following aspects, or in the alternative to some aspects, the side wall can define one or more side flow ports.

In a twenty-ninth aspect, in addition to one or more of the preceding or following aspects, or in the alternative to some aspects, the side wall can define one or more side flow channels.

In a thirtieth aspect, in addition to one or more of the preceding or following aspects, or in the alternative to some aspects, the proximal end wall can include one or more inflow ports.

In a thirty-first aspect, in addition to one or more of the preceding or following aspects, or in the alternative to some aspects, the distal end wall can define one or more outflow ports.

In a thirty-second aspect, in addition to one or more of the preceding or following aspects, or in the alternative to some aspects, the distal end wall does not include an outflow port therein.

In a thirty-third aspect, in addition to one or more of the preceding or following aspects, or in the alternative to some aspects, the fluid dispersal cap can be configured to be attached to the dispersion catheter.

In a thirty-fourth aspect, in addition to one or more of the preceding or following aspects, or in the alternative to some aspects, the fluid dispersal cap can define one or more internal flow conduits.

In a thirty-fifth aspect, in addition to one or more of the preceding or following aspects, or in the alternative to some aspects, the one or more internal flow conduits can be curved.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a dispersion catheter in accordance with various embodiments herein.

FIG. 2 is a schematic view of a pusher wire in accordance with various embodiments herein.

FIG. 3 is a schematic view of components of a dispersion catheter system in accordance with various embodiments herein.

FIG. 4 is a schematic view of a fluid dispersal cap in accordance with various embodiments herein.

FIG. 5 is a schematic view of a dispersion catheter in accordance with various embodiments herein.

FIG. 6 is a schematic view of anatomy relevant for injectate delivery in accordance with various embodiments herein.

FIG. 7 is a schematic view of anatomy and components for injectate delivery in accordance with various embodiments herein.

FIG. 8 is a schematic view of anatomy and components for injectate delivery in accordance with various embodiments herein.

FIG. 9 is a schematic view of anatomy and components for injectate delivery in accordance with various embodiments herein.

FIG. 10 is a schematic view of anatomy and components for injectate delivery in accordance with various embodiments herein.

FIG. 11 is a sectional view of components of a dispersion catheter system in accordance with various embodiments herein.

FIG. 12 is a sectional view of components of a dispersion catheter system in accordance with various embodiments herein.

FIG. 13 is a schematic perspective view of a fluid dispersal cap in accordance with various embodiments herein.

FIG. 14 is a proximal end view of a fluid dispersal cap in in accordance with various embodiments herein.

FIG. 15 is a schematic perspective view of a fluid dispersal cap in accordance with various embodiments herein.

FIG. 16 is a schematic cutaway view of a fluid dispersal cap in accordance with various embodiments herein.

FIG. 17 is a schematic perspective view of a fluid dispersal cap in accordance with various embodiments herein.

FIG. 18 is a proximal end view of a fluid dispersal cap in accordance with various embodiments herein.

FIG. 19 is a schematic perspective view of a fluid dispersal cap in accordance with various embodiments herein.

FIG. 20 is a proximal end view of a fluid dispersal cap in accordance with various embodiments herein.

FIG. 21 is a schematic perspective view of a fluid dispersal cap in accordance with various embodiments herein.

FIG. 22 is a schematic cutaway view of a fluid dispersal cap in accordance with various embodiments herein.

FIG. 23 is a sectional view of components of a dispersion catheter system in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As described above, there are many potential challenges associated with delivering injectates including limited vessel sizes and runway (depending on the target), downstream branching (which could result in delivery to non-target areas), vessel branches in sequence (series), differing flow resistance in some areas, irregular and/or undesirable deposition patterns of composition components, sedation requirements, vasospasm, and catheter positioning inconsistency.

Further delivering therapies (including, but not limited to, embolic therapies and Y 90 radioactive microspheres) to cerebral arteries and prostatic arteries have specific requirements as compared to larger organs like the liver and the kidneys making delivery of injectates to such target sites more challenging. For example, low flow rates can be preferred in cerebral arteries and other small vessels and thus the mixing that may otherwise occur high flow rates and jetting may not be possible. In addition, there may be more branching with some targeted areas such as cerebral and prostatic arteries increasing the chance of some injectate being delivered to a non-target area, which may lead to negative consequences.

Embodiments herein include devices that can enhance dispersion of an injectate with the blood stream, even at relatively slow fluid injection rates. Further, embodiments of devices herein can also reduce reflux flow (backward flow) which may otherwise occur in scenarios where an area of fluid delivery is flooded with injectate. For these and other reasons, devices herein can make interventional treatments safer and more effective. They can also enable new interventional treatments that are currently difficult and/or contraindicated due to an inability to precisely deliver therapeutics to target anatomies.

In various embodiments herein, fluid dispersal caps can cause a flow of fluid to be preferentially diverted away from the centerline of the vessel and at least partially in a radial direction towards the vessel walls. This can provide advantages. First, mixing is enhanced by redistribution of the injectate. The injectate streams first move towards the inner wall of the vessel and then the bulk of the flow moves away from the wall and back towards the central region where the velocities are higher. This results in enhanced entrainment of the injectate by the blood flow as the injectate spreads out. Second, as a relatively low viscosity injectate (in many embodiments) replaces the higher viscosity boundary layer within the vessel, it reduces drag in the vessel due to a lubrication effect of the injectate. With reduced drag, the fluid preferentially passes through the vessel versus backing up within the vessel, reducing chances for backflow and reflux (and potential non-target delivery).

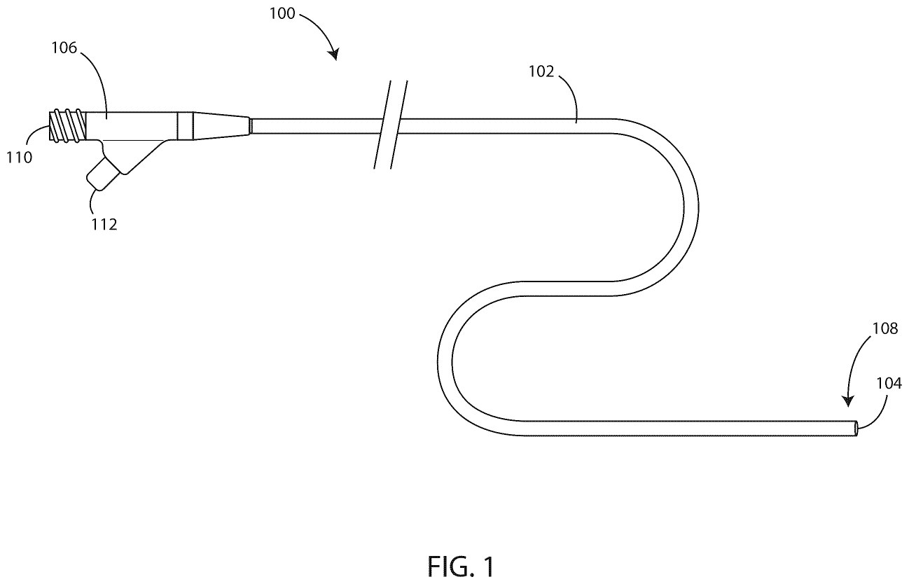

Referring now to FIG. 1, a schematic view is shown of a dispersion catheter 100 in accordance with various embodiments herein. The dispersion catheter 100 can be part of a system for delivering injectates to small diameter vessels of a patient orifice. The dispersion catheter 100 includes a proximal end 106, a dispersion catheter tip 108, and a dispersion catheter shaft 102 disposed in between. The dispersion catheter shaft 102 and/or other components of the dispersion catheter can be formed of various materials including various polymers. In some embodiments, the dispersion catheter shaft 102 and/or other components of the dispersion catheter can be formed with polymers including polyether block amide copolymer (PEBAX), polyethylenes (low-density and high-density), polyamides, thermoplastic polyurethanes, polypropylene, various fluoropolymers, polyvinyl chloride, and the like.

In this example, the proximal end 106 can include an adapter such as a Y connector as well as various ports such as a wire port 110 (for guidewires or positioning or pusher wires herein), an injectate port 112, and the like. However, it will be appreciated that this is just one example and that various other features to facilitate connection with other devices can be included at the proximal end 106. The dispersion catheter 100 also includes an end hole 104 or outflow orifice which is in fluid communication with a lumen of the dispersion catheter 100.

In various embodiments herein, the dispersion catheter shaft 102 can have a relatively small diameter to facilitate delivery of an injectate to small diameter target vessels. For example, the dispersion catheter shaft 102 can have an outside diameter of less than 1, 0.8, 0.6, 0.5, 0.4, or 0.3 millimeters (mm) (0.039, 0.031, 0.024, 0.020, 0.016, or 0.012 inches (in)), or an outside diameter falling within a range between any of the forgoing.

The dispersion catheter shaft 102 can define a lumen therein (not shown in this view). The dispersion catheter tip 108 can have various different geometries. The dispersion catheter tip 108 can be in fluid communication with the lumen of a dispersion catheter shaft 102. In operation, an injectate can be inserted into the dispersion catheter 100 through an injectate port 112 in the proximal end 106 thereof and pass through the lumen of the dispersion catheter shaft 102 before reaching the end hole 104. A fluid dispersal cap (described further below) can be configured to be disposed within a path of fluid flow. In some embodiments, the fluid dispersal cap can be positioned fully inside of the end hole 104 of the dispersion catheter tip 108, at least partially outside of the end hole 104, or fully outside of the end hole 104.

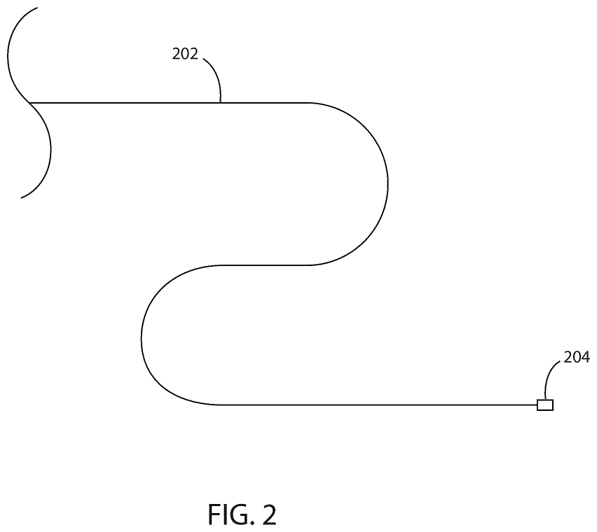

In some embodiments, the fluid dispersal cap can be attached to the dispersion catheter, such as near the catheter tip 108. Referring now to FIG. 2, a schematic view of a wire 202 (in this case a delivery wire for the fluid dispersal cap) is shown in accordance with various embodiments herein. In this example, the wire 202 is attached to a fluid dispersal cap 204 at a distal end of the wire 202. The wire 202 can be formed of a biocompatible metal such as stainless steel, various steel alloys, titanium, titanium alloys, nitinol, and the like. In some cases, the wire 202 can be coated with another material (such as various polymers or the like) for biocompatibility, lubricity, or the like. The fluid dispersal cap 204 can be configured to be disposed within a path of fluid flow coming from a dispersion catheter tip 108 and the wire 202 can be used to position the fluid dispersal cap 204, such as by moving the wire 202 in or out relative to the fluid dispersal catheter (shown in FIG. 1). As such, the delivery wire 202 can be configured to pass through the lumen of the dispersion catheter shaft.

The fluid dispersal cap 204 can be formed of various materials including, but not limited to, various polymers, metals, composites, or even glasses or ceramics. In some embodiments, the fluid dispersal cap 204 can be formed of a material facilitating its attachment to the wire 202 (in the embodiment of FIG. 2) or its attachment to other components such as the fluid dispersal catheter itself in other embodiments herein. In the embodiment of FIG. 1, the fluid dispersal cap 204 can be formed of a biocompatible metal and can be attached to the wire 202 using techniques such as welding, brazing, soldering, adhesive attachment, or even mechanical attachment. The fluid dispersal cap 204 can be formed using various techniques including one or more of additive manufacturing techniques, milling techniques, casting, and the like.

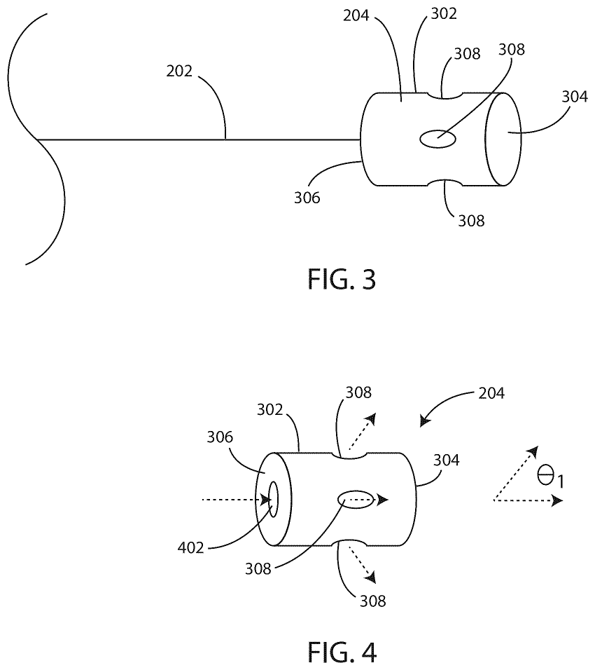

The fluid dispersal cap 204 can include various features in order to promote the dispersal of injectates delivered through the system. Referring now to FIG. 3, a schematic view of components of a dispersion catheter system is shown in accordance with various embodiments herein. FIG. 3 shows a wire 202 connected to the fluid dispersal cap 204. The fluid dispersal cap 204 includes a side wall 302, a distal end wall 304, and a proximal end wall 306. In various embodiments, the side wall 302 interconnects the proximal end wall 306 and the distal end wall 304. The side wall 302 can define one or more side flow ports 308 (e.g., 1, 2, 3, 4, 5, 6, 7, 8 or more, or a number falling within a range between any of the foregoing). The side flow ports 308 can take on various shapes including circular, oval, irregular, or the like. In some embodiments, the side flow ports 308 can be positioned symmetrically around the side wall 302 perimeter of the fluid dispersal cap 204, but in some embodiments can be positioned asymmetrically. In some embodiments, the side flow ports 308 can be positioned such that they are closer to the distal end wall 304 than to the proximal end wall 306. However, in other embodiments, the side flow ports 308 can be approximately equidistant between the distal end wall 304 and the proximal end wall 306, or even disposed closer to the proximal end wall. In various embodiments, the fluid dispersal cap 204 can be circular or semicircular in cross-section, however other shapes are also contemplated herein. In various embodiments, the distal end wall 304 does not include an outflow port thereon such as shown in FIG. 3, however, in some other embodiments herein the distal end wall 304 can include one or more outflow ports.

The side flow ports of the fluid dispersal cap 204 can redirect a flow of an injectate from a first direction along a lengthwise axis of the catheter shaft to one or more radial directions such that the flow is directed at least partially toward luminal walls of the vessel in which the dispersal catheter is disposed. Referring now to FIG. 4, a schematic view of a fluid dispersal cap 204 is shown in accordance with various embodiments herein. As before, the fluid dispersal cap 204 includes a side wall 302, a distal end wall 304, and a proximal end wall 306 and the side wall 302 defines one or more side flow ports 308. In this view, it can be seen that the proximal end wall 306 includes one or more inflow ports 402. Such inflow ports 402 can be centrally positioned on the proximal end wall 306. However, as shown in some other examples herein, inflow ports can be positioned in other places. For example, inflow ports can be non-centrally positioned, offset from the center, closer to the perimeter of the proximal end wall 306 than the center thereof, etc. In some embodiments, the inflow port(s) 402 can have a cross-sectional surface area at the point where it intersects the proximal end wall 306 that is about 5, 10, 15, 20, 25, 30, 40, 50, 60, 70, 80, 90, or 95 percent or higher of the surface area of the proximal end wall 306, or an amount falling within a range between any of the foregoing.

At least some of the flow of the injectate passing through the dispersion catheter enters the inflow port 402 (or ports) and exits the side flow ports 308 with the direction of flow having been changed to a more radial direction. In some embodiments, 100% of the injectate passes through the inflow port(s) 402. However, in other embodiments, such as depending on the positioning of the fluid dispersal cap 204, less than 100% of the injectate passes through the inflow port(s) 402, as some may bypass the fluid dispersal cap 204 and/or in some embodiments, the fluid dispersal cap 204 may lack inflow ports (see embodiment described below) entirely. In some embodiments the amount of fluid passing through the fluid dispersal cap 204 can be equal to 100%, 90%, 80%, 70%, 60%, 50%, 40%, 30%, 20%, 10%, 5%, or even 0% (such as when there are no inflow ports) of the total injectate volume, or an amount falling within a range between any of the foregoing.

As referenced above, the direction of flow can be changed to a more radial direction. In some embodiments, the main direction of flow can be changed (with respect to the direction of a lengthwise axis of the dispersion catheter) by an angle θ₁ (theta one) of at least about 5, 10, 15, 20, 25, 30, 35, 40, 45, 50, 55, 60, 65, 70, 75, 80, 85, or even 90 degrees, or an angle falling within a range between any of the foregoing.

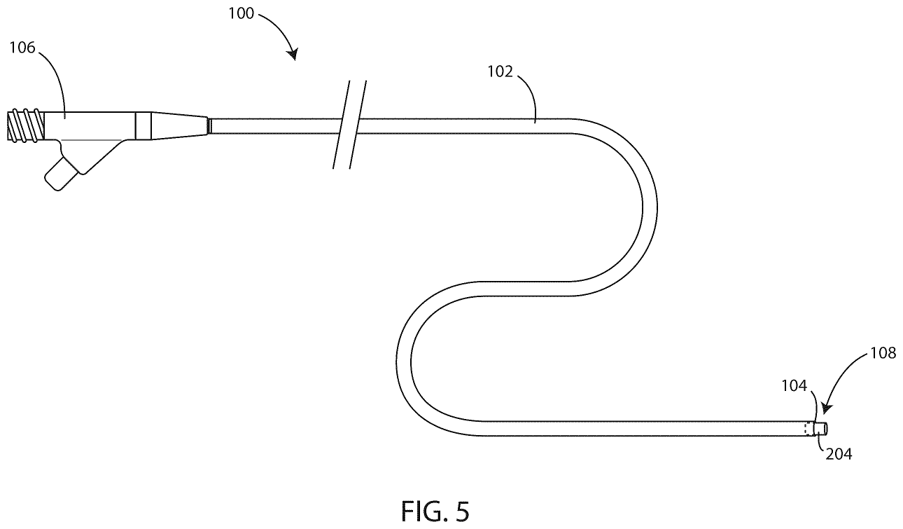

In some embodiments the fluid dispersal cap can be attached to a wire used for positioning of the fluid dispersal cap and in other embodiments the fluid dispersal cap can be attached to the dispersion catheter directly. Regardless, in some embodiments, the fluid dispersal cap can be positioned to be fully within the end of the dispersion catheter, fully outside the end of the dispersion catheter, or partially in and partially out. Referring now to FIG. 5, a schematic view of a dispersion catheter 100 is shown in accordance with various embodiments herein. As before, the dispersion catheter includes a proximal end 106, a catheter shaft 102 (defining a lumen therein), and a dispersion catheter tip 108 with an end hole 104. In this example, the fluid dispersal cap 204 is illustrated positioned at least partially inside of the dispersion catheter tip 108, but extending out therefrom.

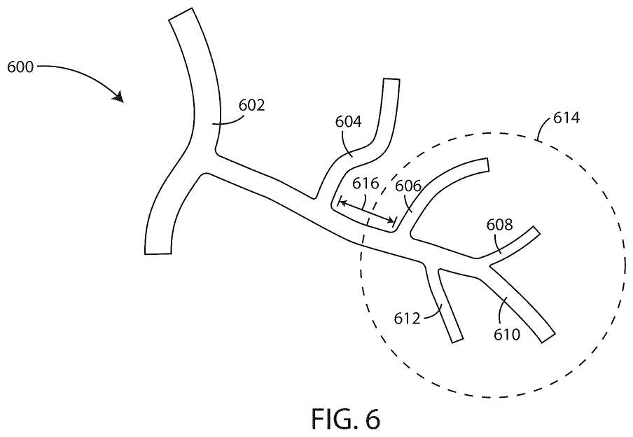

It will be appreciated that dispersion catheter systems and devices herein can address issues associated with delivering injectates and, in particular, delivering injectates to small diameter vessels. For example, embodiments herein can enhance dispersion of an injectate with the blood stream and/or can also reduce reflux flow. Referring now to FIG. 6, a schematic view of vessel anatomy relevant for injectate delivery is shown in accordance with various embodiments herein. The vessel anatomy 600 shown includes a small diameter access vessel 602. The vessel anatomy 600 also includes a side branch vessel 604. In this example, the side branch vessel 604 can lead to a non-target area or organ and thus it is desirable to not deliver an injectate into the side branch vessel 604. The vessel anatomy 600 also includes a feeder vessel target area 614. The feeder vessel target area 614 includes vessels that lead to the target area or organ and thus it is desirable to deliver as much of the total amount of injectate into the feeder vessel target area 614 as possible. In this example, the feeder vessel target area 614 includes a first feeder vessel 606, a second feeder vessel 608, a third feeder vessel 610, and a fourth feeder vessel 612. It will be appreciated, however, that this is only an example and that the feeder vessel target area 614 can include any specific number of feeder vessels.

In this example, the distance 616 from the side branch vessel 604 (leading to a non-target area) to the first feeder vessel 606 is less than one centimeter. This relatively small distance can cause a challenge with respect to injectate delivery because reflux flow, if it occurs, could cause some of the injectate to pass into the side branch vessel 604 even if the injectate is injected at a point past where the side branch vessel 604 branches off. However, devices herein can reduce issues of reflux flow and thus reduce or even eliminate injectate from entering the side branch vessel 604.

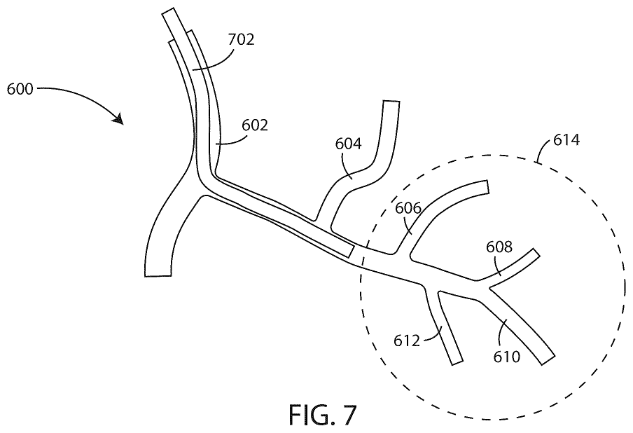

Various operations can be performed when delivering injectates using systems and devices herein. One approach is described with reference to FIGS. 7 to 10. However, it will be appreciated that a greater or lesser number of operations can be performed in various embodiments. Referring now to FIG. 7, a schematic view of vessel anatomy 600 and components for injectate delivery is shown in accordance with various embodiments herein. The vessel anatomy 600 is as described with respect to FIG. 6 and includes a small diameter access vessel 602, a side branch vessel 604, and a feeder vessel target area 614 along with the feeder vessels 606, 608, 610, and 612 therein. FIG. 7 also shows a microcatheter 702. The microcatheter 702 has been advanced within the vessel anatomy 600, and specifically through the small diameter access vessel 602 and past the side branch vessel 604 at a point just upstream from the vessel target area 614. The microcatheter 702 can have an inner diameter that is sufficiently large to accommodate a dispersion catheter 100 herein. The microcatheter 702 can have an outer diameter that is sufficiently small so as to be able to pass through relatively small vessels to get to a target area of the vasculature. In various embodiments, the outside diameter of the microcatheter 702 is less than 3, 2.5, 2, 1.5 or 1 mm (0.118, 0.098, 0.079, 0.059, 0.039 in), or a diameter falling within a range between any of the foregoing. In an embodiment of the method, the microcatheter has an inner diameter or less than 0.06, 0.05, 0.04, 0.03, or even 0.02 inches (1.52, 1.27, 1.02,.076, or 0.51 mm respectively), or an inner diameter falling within a range between any of the foregoing such as from 0.021 to 0.027 inches (0.53 mm to 0.68 mm).

The microcatheter 702 can be formed of various materials including, for example, the polymers described above with respect to the dispersion catheter.

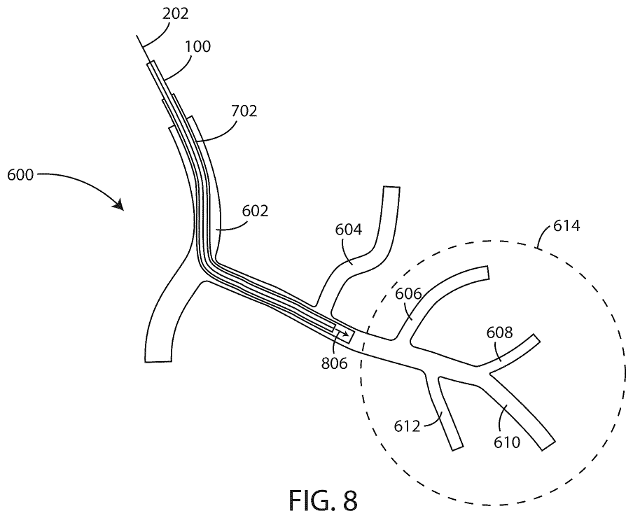

Next, the dispersion catheter can be inserted and advanced into position. To illustrate this, FIG. 8 shows a schematic view of vessel anatomy 600 and components for injectate delivery in accordance with various embodiments herein. As before, the vessel anatomy 600 includes a small diameter access vessel 602, a side branch vessel 604, and a feeder vessel target area 614 along with the feeder vessels 606, 608, 610, and 612 therein. FIG. 8 shows a microcatheter 702 already having been advanced into position and a dispersion catheter 100 being advanced through the microcatheter 702. In some embodiments, the dispersion catheter 100 can be advanced along with the aid of a wire 202 (in the form of a pusher wire). The wire 202 (in this embodiment) can be sufficiently stiff so as to function as a pusher wire transmitting force to the dispersion catheter 100 and yet flexible enough to pass through the vasculature to get to the target area. In this illustration, the dispersion catheter 100 is advancing in direction 806. In some embodiments, the dispersion catheter 100 can be advanced until it reaches the open distal end of the microcatheter 702. However, in other embodiments, it can be advanced to a position short of the distal end of the microcatheter 702 or even past the distal end of the microcatheter 702. In this example, the dispersion catheter 100 is advanced to a point past the side branch vessel 604.

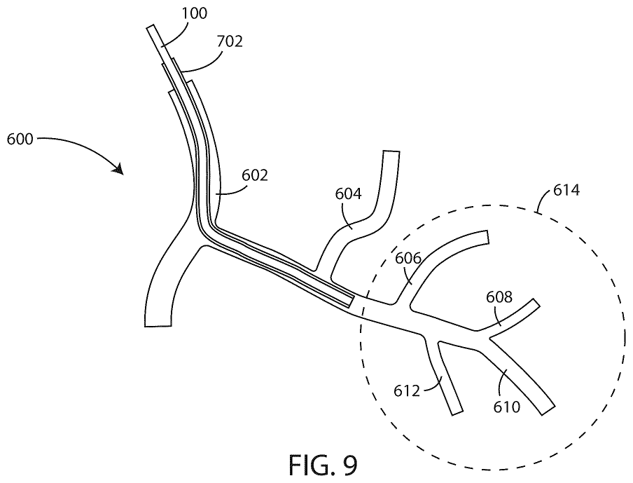

Next, the wire 202 can be withdrawn. Referring now to FIG. 9, a schematic view of vessel anatomy 600 and components for injectate delivery is shown in accordance with various embodiments herein. As before, the vessel anatomy 600 includes a small diameter access vessel 602, a side branch vessel 604, and a feeder vessel target area 614 along with the feeder vessels 606, 608, 610, and 612 therein. FIG. 9 also shows the microcatheter 702 and the dispersion catheter 100 positioned within the same. In this example, the wire 202 (not shown in this view) has been withdrawn leaving the dispersion catheter in a desired position. However, it will be appreciated that in some embodiments it can be possible to leave the wire in place.

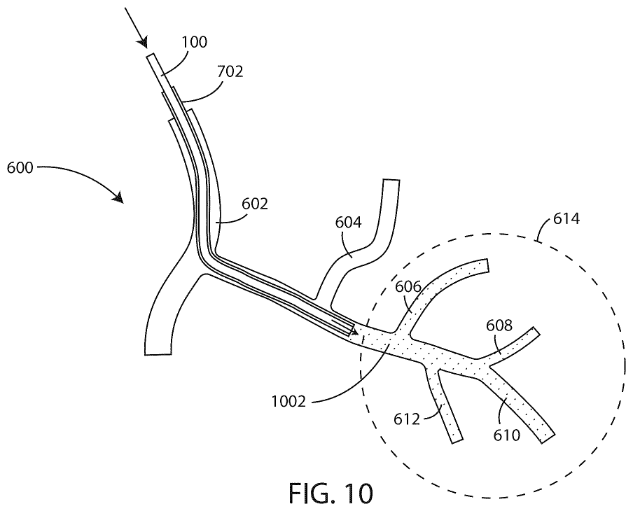

Next, the injectate can be delivered. Referring now to FIG. 10, a schematic view of vessel anatomy 600 and components for injectate 1002 delivery is shown in accordance with various embodiments herein. As before, the vessel anatomy 600 includes a small diameter access vessel 602, a side branch vessel 604, and a feeder vessel target area 614 along with the feeder vessels 606, 608, 610, and 612 therein. FIG. 10 also shows the microcatheter 702 and the dispersion catheter 100 positioned within the same. The injectate 1002 can be injected into a port at the proximal end (not shown in this view) of the dispersion catheter and flow through the same toward the catheter tip thereof. FIG. 10 also shows an injectate 1002 being delivered to the feeder vessel target area 614, with the flow thereof being impacted by a fluid dispersal cap (not shown in this view).

In some embodiments, the flow rate of the injectate can be less than or equal to 20, 15, 12.5, 10, 7.5, 6, or even 5 milliliters (mL)/minute, or a flow rate falling within a range between any of the foregoing.

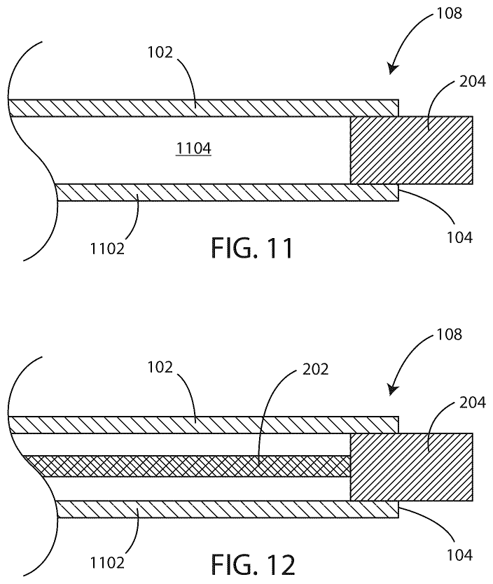

As referenced above, the fluid dispersal cap 204 can be disposed within the distal end of catheter shaft in some embodiments herein. Referring now to FIG. 11, a sectional view is shown of components of a dispersion catheter 100 system in accordance with various embodiments herein. FIG. 11 shows a dispersion catheter shaft 102 along with a shaft wall 1102 and a lumen 1104 defined therein. FIG. 11 also shows the fluid dispersal cap 204 that can be configured to be attached to the dispersion catheter shaft 102. In particular, the fluid dispersal cap 204 can be within the end hole 104 at the dispersion catheter tip 108 of the dispersion catheter. It will be appreciated that the fluid dispersal cap 204 can be secured in place in various ways. For example, in some embodiments, the fluid dispersal cap 204 can be held in place within the dispersion catheter tip 108 using an adhesive. In some embodiments, the fluid dispersal cap 204 can be held in place by friction or using mechanical device to secure the same.

In contrast to the embodiment of FIG. 11, as referenced above in some embodiments, the fluid dispersal cap 204 is secured to a wire and the wire is used to position the fluid dispersal cap as desired. Referring now to FIG. 12, a sectional view of components of a dispersion catheter system is shown in accordance with various embodiments herein. FIG. 12 shows dispersion catheter shaft 102 along with a shaft wall 1102 and a lumen 1104 defined therein. FIG. 12 also shows fluid dispersal cap 204 within the end hole 104 at the dispersion catheter tip 108 of the dispersion catheter. However, in this embodiment, the fluid dispersal cap 204 is attached to a wire 202 (a positioning wire) and the wire 202 holds the fluid dispersal cap 204 in a desired position with respect to the end hole 104 of the dispersion catheter shaft 102. In some embodiments the wire 202 can be attached to a central portion of proximal end wall of the fluid dispersal cap 204. In such as case, any inflow ports on the proximal end wall can be disposed to the outside of where the wire 202 is attached. However, in some embodiments, the wire 202 may be attached to the proximal end wall off center and/or the wire 202 can split into portions to be attached outside the central portion of the proximal end wall of the fluid dispersal cap 204, but symmetrically about the central portion. This positioning can facilitate central placement of any inflow ports.

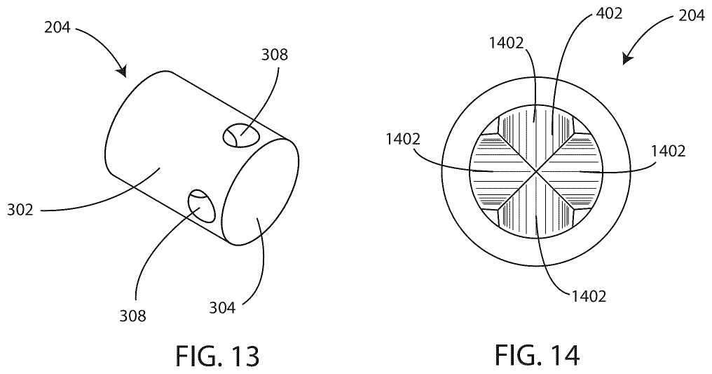

It will be appreciated that fluid dispersal caps herein can take many different forms. FIGS. 13-21 illustrate some of these forms. Referring now to FIG. 13, a schematic perspective view is shown of a fluid dispersal cap 204 in accordance with various embodiments herein. The fluid dispersal cap 204 includes a side wall 302, a distal end wall 304, and defines one or more side flow ports 308. The fluid dispersal cap 204 of FIG. 13 can be effective to reduce or eliminate fluid jetting. Referring now to FIG. 14, a proximal end view of the fluid dispersal cap 204 of FIG. 13 is shown in accordance with various embodiments herein. The fluid dispersal cap 204 includes inflow port 402 and one or more flow diverter surfaces 1402 therein. The diverter surfaces 1402 can serve to direct the fluid flow into one or more internal conduits or passages (not shown in this view) through the fluid dispersal cap 204 before exiting one or more side flow ports 308.

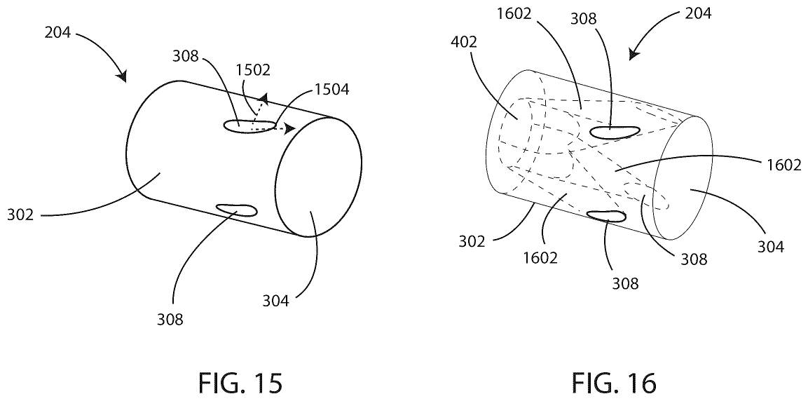

Referring now to FIG. 15, a schematic perspective view of a fluid dispersal cap 204 is shown in accordance with various embodiments herein. The fluid dispersal cap 204 includes a side wall 302, a distal end wall 304, and defines one or more side flow ports 308. The fluid dispersal cap 204 of FIG. 15 can be effective to reduce or eliminate fluid jetting. Referring now to FIG. 16, a schematic cutaway view of the fluid dispersal cap of FIG. 15 is shown in accordance with various embodiments herein. The fluid dispersal cap 204 can define one or more internal flow conduits 1602. The internal flow conduits 1602 can provide a passage for fluid to flow from the one or more inflow ports 402 to the one or more side flow ports 308. In this embodiment, the one or more internal flow conduits 1602 are curved. In some embodiments, the internal flow conduits 1602 can be shaped such that they direct the flow at least partially in a radial direction (such as towards luminal surfaces of the vessel in which the injectate is being delivered) and/or be pointed at least partially in a direction of a tangent line 1502 to the outer radial circumference of the fluid dispersal cap such as illustrated by arrow 1504 of FIG. 15, which can cause the fluid flow to swirl in a clockwise or counter-clockwise swirl creating a vortex within the vessel in which the injectate is delivered.

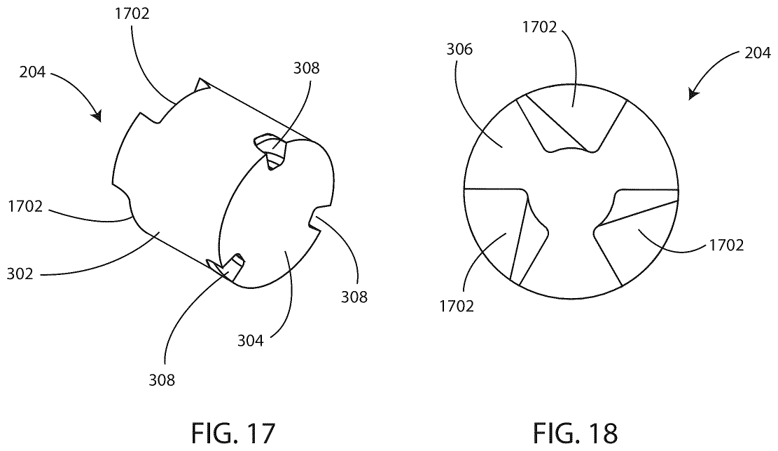

Referring now to FIG. 17, a schematic perspective view is shown of another example of a fluid dispersal cap in accordance with various embodiments herein. The fluid dispersal cap 204 includes a side wall 302, a distal end wall 304, and one or more side flow ports 308. In this example, a proximal end wall of the fluid dispersal cap includes one or more edge inflow ports 1702 (e.g., inflow ports contacting a peripheral edge of the proximal end wall). In this example, the side flow ports 308 can contact both the side wall 302 as well as a peripheral edge of the distal end wall 304. The fluid dispersal cap 204 of FIG. 17 can be effective to reduce or eliminate fluid jetting.

Referring now to FIG. 18, a proximal end view is shown of the fluid dispersal cap of FIG. 17. In this view, proximal end wall 306 of the fluid dispersal cap 204 is shown. The proximal end wall 306 includes edge inflow ports 1702.

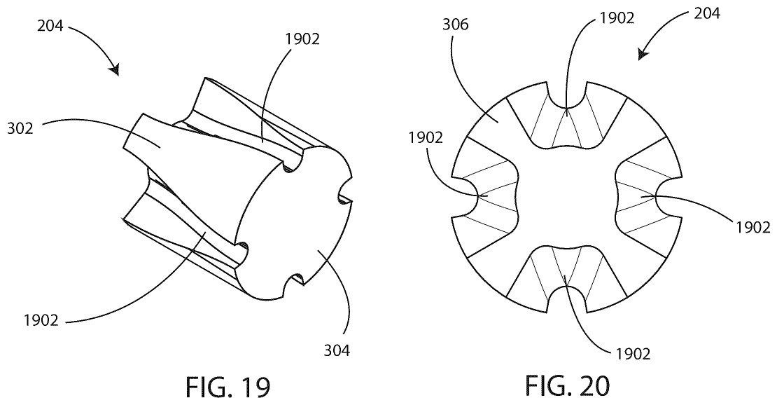

Referring now to FIG. 19, a schematic perspective view is shown of another example of a fluid dispersal cap in accordance with various embodiments herein. The fluid dispersal cap 204 includes a side wall 302 and a distal end wall 304. In this example, the side wall 302 defines one or more side flow channels 1902 that remain open to the exterior of the side wall 302. For example, the side flow channels 1904 can be open around the exterior radial perimeter of the fluid dispersal cap 204. In this way, a flow path is formed between the side flow channels 1902 and an interior surface of the dispersal catheter into which the fluid dispersal cap 204 is placed. The fluid dispersal cap 204 of FIG. 19 can be effective to reduce or eliminate fluid jetting. Referring now to FIG. 20, a proximal end 106 view of the fluid dispersal cap of FIG. 19 is shown in accordance with various embodiments herein. The fluid dispersal cap 204 includes a proximal end wall 306 and the proximal ends of the one or more side flow channels 1902 are shown intersecting with a proximal end wall 306.

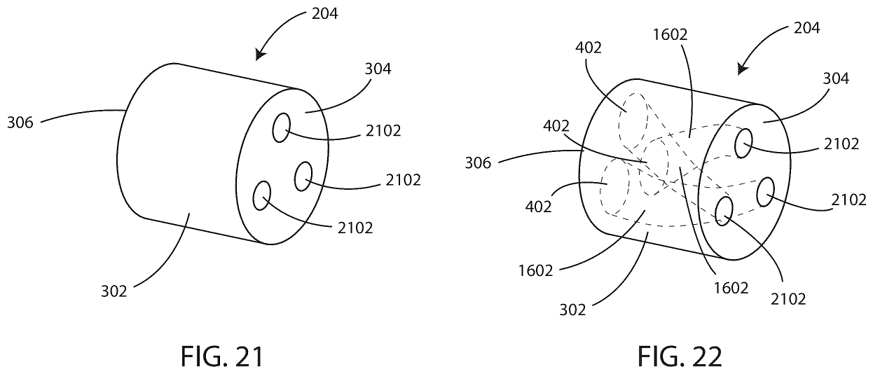

Referring now to FIG. 21, a schematic perspective view is shown of another example of a fluid dispersal cap 204 in accordance with various embodiments herein. The fluid dispersal cap 204 includes a side wall 302, a distal end wall 304, and a proximal end wall 306. In this example, the distal end wall 304 can define a plurality of outflow ports 2102. Referring now to FIG. 22, a schematic cutaway view of the example fluid dispersal cap 204 of FIG. 21 is shown include various internal features thereof. As before, the fluid dispersal cap 204 includes a side wall 302, a distal end wall 304, and a proximal end wall 306. In this view, it can be seen that the fluid dispersal cap 204 also defines one or more internal flow conduits 1602 or flow paths. The internal flow conduits 1602 can be in fluid communication with the outflow ports 2102. Also, it can be seen that the proximal end wall 306 includes one or more inflow ports 402. In this example, the one or more internal flow conduits 1602 are curved, such that the inflow ports 402 are disposed at different positions on the face of the proximal end wall 306 than their corresponding outflow ports 2102 are on the face of the distal end wall 304. For example, the internal flow conduits 1602 can have a partial helical or twisted shape resulting in twisting the fluid flow and/or fluid flow with a clockwise or counterclockwise swirl creating a vortex after exiting the fluid dispersal cap 204. The fluid dispersal cap 204 of FIG. 22 can be effective to reduce or eliminate fluid jetting.

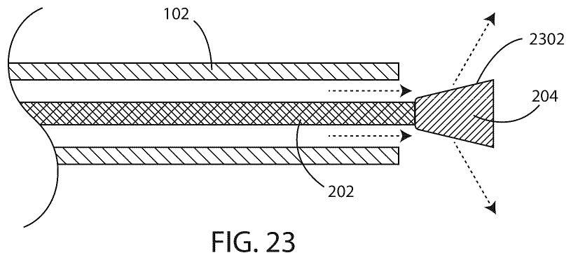

In some embodiments, the proximal end wall and the distal end wall can be approximately the same outer dimensions. However, in other embodiments, the proximal end wall can be smaller than the distal end wall in out dimensions such as in the context of a conical shape. Referring now to FIG. 23, a sectional view is shown of components of a dispersion catheter system in accordance with various embodiments herein. FIG. 23 shows a dispersion catheter shaft 102 along with a wire 202 (in the form of a positioning wire) disposed therein. The fluid dispersal cap 204 is attached to the wire 202. In this example, the fluid dispersal cap 204 includes a conical body member 2302, with the small end of the conical body member 2302 upstream such that a fluid flow that impinges on the surface of the body member 2302 is pushed outward in a radial direction.

### Methods

Many different methods are contemplated herein, including, but not limited to, methods of making a dispersion catheter system, methods of delivering an injectate, methods of delivering an injectate to a small diameter vessel, methods of dispersing an injectate, and the like. Aspects of system/device operation described elsewhere herein can be performed as operations of one or more methods in accordance with various embodiments herein.

In an embodiment, a method of delivering an injectate to a small diameter vessel of a patient is included. The method can include inserting a microcatheter into a vessel of the patient. The method can also include advancing a dispersion catheter to an end of the microcatheter using a pusher wire. The dispersion catheter can include a fluid dispersal cap. The method can also include removing the pusher wire from the dispersion catheter.

The method can also include delivering a therapeutic composition to the patient through the dispersion catheter.

In an embodiment of the method, the microcatheter has an inner diameter or less than 0.06, 0.05, 0.04, 0.03, or even 0.02 inches (1.52, 1.27, 1.02,.076, or 0.51 mm respectively), or an inner diameter falling within a range between any of the foregoing such as from 0.021 to 0.027 inches (0.53 mm to 0.68 mm). In an embodiment of the method, the dispersion catheter has an outside diameter of less than 1, 0.8, 0.6, 0.5, 0.4, or 0.3 mm (0.039, 0.031, 0.024, 0.020, 0.016, or 0.012 in), or an outside diameter falling within a range between any of the forgoing.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A dispersion catheter system for delivering injectates to small diameter vessels of a patient comprising:
a dispersion catheter shaft, the dispersion catheter shaft defining a lumen;
a dispersion catheter tip, wherein the dispersion catheter tip is in fluid communication with the lumen of the dispersion catheter shaft; and
a fluid dispersal cap, wherein the fluid dispersal cap is configured to be disposed within a path of fluid flow coming from the dispersion catheter tip.

2. The catheter system of claim 1, wherein the dispersion catheter shaft has an outside diameter of less than 0.039 in (1 mm).

3. The catheter system of claim 1, wherein the fluid dispersal cap is positioned at least partially inside of an outflow orifice.

4. The catheter system of claim 1, wherein the fluid dispersal cap is positioned fully outside of the dispersion catheter tip.

5. The catheter system of claim 1, the fluid dispersal cap comprising:
a proximal end wall;
a distal end wall; and
a side wall, wherein the side wall interconnects the proximal end wall and the distal end wall.

6. The catheter system of claim 5, the side wall defining one or more side flow ports.

7. The catheter system of claim 6, wherein the one or more side flow ports are configured to direct a flow of fluid at least partly in a radial direction of the fluid dispersal cap.

8. The catheter system of claim 5, the side wall defining one or more side flow channels.

9. The catheter system of claim 5, the proximal end wall comprising one or more inflow ports.

10. The catheter system of claim 9, wherein the one or more inflow ports are centrally positioned on the proximal end wall.

11. The catheter system of claim 9, wherein the one or more inflow ports are positioned around the periphery of the proximal end wall.

12. The catheter system of claim 5, the distal end wall defining one or more outflow ports.

13. The catheter system of claim 12, wherein the one or more outflow ports contact a peripheral edge of the distal end wall.

14. The catheter system of claim 5, wherein the distal end wall does not include an outflow port therein.

15. The catheter system of claim 1, the fluid dispersal cap defining one or more internal flow conduits.

16. The catheter system of claim 15, wherein the one or more internal flow conduits are curved.

17. The catheter system of claim 15, wherein the one or more internal flow conduits are at least partly helical.

18. The catheter system of claim 1, wherein the fluid dispersal cap is configured to be attached to the dispersion catheter shaft.

19. The catheter system of claim 1, further comprising a delivery wire;
wherein the fluid dispersal cap is configured to be attached to a distal end of the delivery wire; and
wherein the delivery wire is configured to pass through the lumen of the dispersion catheter shaft.

20. A method of delivering an injectate to a small diameter vessel of a patient comprising:
inserting a microcatheter into a vessel of the patient; and
advancing a dispersion catheter to an end of the microcatheter using a pusher wire, the dispersion catheter comprising a fluid dispersal cap;
removing the pusher wire from the dispersion catheter; and
delivering a therapeutic composition to the patient through the dispersion catheter.
